(19) United States
(12) Patent Application Publication
(10) Pub. No.: US 2018/0263538 A1
(43) Pub. Date: Sep. 20, 2018

(54) DYNAMIC SWEAT SENSING DEVICE MANAGEMENT

(71) Applicant: Eccrine Systems, Inc., Cincinnati, OH (US)

(72) Inventors: Jason Heikenfeld, Cincinnati, OH (US); Robert Beech, Cincinnati, OH (US)

(73) Assignee: Eccrine Systems, Inc., Cincinnati, OH (US)

(21) Appl. No.: 15/553,210

(22) PCT Filed: Feb. 24, 2016
(86) PCT No.: PCT/US16/19282

§ 371 Date: Aug. 24, 2017

Related U.S. Application Data
(60) Provisional application No. 62/120,342, filed on Feb. 24, 2015.

(51) Int. Cl. A61B 5/145; A61B 5/00

(52) U.S. Cl. CPC A61B 5/14517; A61B 5/0531; A61B 2560/0204; A61B 5/6843

(57) ABSTRACT

The disclosure provides: a two-way communication means between a sweat sensing device and a user; at least one means of activating, deactivating, controlling the sampling rate, and controlling the electrical power applied to a particular sweat sensor or group of sensors; a means of isolating a sweat sensor from sweat until needed; a means of selectively stimulating sweat for a particular sweat sensor or group of sensors to manage sweat flow or generation rate; a means of monitoring the power consumption of a sensor device, individual sensors or groups of sensors; a means of monitoring an individual sweat sensor or group of sensors for optimal performance; a means of monitoring whether a sweat sensing patch is in adequate proximity to a wearer's skin to allow device operation; and the ability to use aggregated sweat sensor data correlated with external information to enhance the device's management capabilities.

### CROSS-REFERENCE TO RELATED APPLICATIONS

The present application relates to U.S. Provisional Application No. 62/120,342, filed Feb. 24, 2015, and has specification that builds upon PCT/US14/061098, filed Oct. 17, 2014; and PCT/US15/55756, filed Oct. 15, 2015, the disclosures of which are hereby incorporated herein by reference in their entirety.

### STATEMENT REGARDING FEDERALLY SPONSORED RESEARCH OR DEVELOPMENT

No federal funds were utilized for this invention.

### BACKGROUND OF THE INVENTION

Sweat sensing technologies have enormous potential for applications ranging from athletics, to neonatology, to workforce safety, to pharmacological monitoring, to personal digital health, to name a few applications. Sweat contains many of the same biomarkers, chemicals, or solutes that are carried in blood, which can provide significant information enabling one to diagnose illnesses, health status, exposure to toxins, performance, and other physiological attributes even in advance of any physical sign. Furthermore sweat itself, the action of sweating, and other parameters, attributes, solutes, or features on, near, or beneath the skin, can be measured to further reveal physiological information.

Of all the other physiological fluids used for bio monitoring (e.g., blood, urine, saliva, tears), sweat has arguably the most variable sampling rate as its collection methods and variable rate of generation both induce large variances in the effective sampling rate. Sweat also contains concentrations of solutes that are highly variable over time, depending not just on the concentration of those solutes in the blood, but also on eccrine sweat gland function. Further, a sweat sensor may experience significant variation in the level of proper contact with the skin or the sweat sample, which can cause variations capable of corrupting useful data. These factors unique to sweat sampling pose a significant challenge to accurate, reliable sweat readings, especially in continuous monitoring applications.

Sweat has significant potential as a sensing paradigm, but it has not emerged beyond decades-old usage in infant chloride assays for Cystic Fibrosis (e.g. Wescor Macroduct system) or in illicit drug monitoring patches (e.g. PharmCheck drugs of abuse patch by PharmChem). The majority of medical literature discloses slow and inconvenient sweat stimulation and collection, transport of the sample to a lab, and then analysis of the sample by a bench-top machine and a trained expert. All of this is so labor intensive, complicated, and costly, that in most cases, one would just as well implement a blood draw, since it is the gold standard for most forms of high performance biomarker sensing. Hence, sweat sensing has not achieved its fullest potential for biosensing, especially for continuous or repeated biosensing or monitoring. Furthermore, attempts at using sweat to sense “holy grails” such as glucose have failed to produce viable commercial products, reducing the publically perceived capability and opportunity space for sweat sensing. A similar conclusion has been made very recently in a substantial 2014 review provided by Castro titled “Sweat: A sample with limited present applications and promising future in metabolomics,” which states: “The main limitations of sweat as clinical sample are the difficulty to produce enough sweat for analysis, sample evaporation, lack of appropriate sampling devices, need for a trained staff, and errors in the results owing to the presence of pilocarpine. In dealing with quantitative measurements, the main drawback is normalization of the sampled volume.”

Many of the drawbacks stated above can be resolved by creating novel and advanced interplays of chemicals, materials, sensors, electronics, microfluidics, algorithms, computing, software, systems, and other features or designs, in a manner that affordably, effectively, conveniently, intelligently, or reliably brings sweat sensing technology into intimate proximity with sweat as it is generated.

Of particular interest is the ability to dynamically control sweat sensors in real time in order to reduce power consumption by the sweat sensing device, to optimize sensor lifespan and performance, to enable the use of limited lifespan sensors, and to manage skin or sweat contact issues.

### SUMMARY OF THE INVENTION

The present disclosure is premised on the realization that sweat can be effectively stimulated and analyzed in a single, continuous, or repeated manner inside the same device. The disclosed invention addresses the confounding difficulties involving such analysis by enabling sweat sensors to be dynamically controlled in real time in order to reduce power consumption by the sweat sensing device, to optimize sensor lifespan and performance, to enable the use of limited lifespan sensors, and to manage skin or sweat contact issues. Specifically, the disclosed invention provides: at least one component capable of facilitating two-way communication between a sweat sensing device and a device user; at least one means of activating, deactivating, controlling the sampling rate, and controlling the electrical power applied to a particular sweat sensor or group of sensors on the device; a means of isolating a sweat sensor from sweat or power until its capabilities are needed; a means of selectively stimulating sweat for a particular sweat sensor or group of sensors to manage sweat flow or sweat generation rate; a means of monitoring the power consumption of a sweat sensor device, individual sensors or groups of sensors; a means of monitoring an individual sweat sensor or group of sensors for optimal performance; a means of monitoring whether a sweat sensing patch is in adequate contact with or proximity to a wearer's skin to allow device start-up and operation; and the ability to use aggregated sweat sensor data that may be correlated with information external to the sweat sensing device to enhance the device's dynamic management capabilities.

### BRIEF DESCRIPTION OF THE DRAWINGS

The objects and advantages of the present disclosure will be further appreciated in light of the following detailed descriptions and drawings in which:

FIG. 1 is a generic representation of the disclosed invention including a mechanism for stimulating and analyzing sweat sensor data on a singular, continuous or repeated basis.

FIG. 2 is an example embodiment of at least a portion of a device of the present disclosure including a mechanism for generating sweat sensor data that may be used to inform dynamic control of a sweat sensor or group of sensors.

FIG. 3 is an example embodiment of at least a portion of the present disclosure including a mechanism for gating a single use or limited use sweat sensor from a sweat sample.

FIG. 4 is an example embodiment of at least a portion of a device of the present disclosure including a mechanism for determining adequate skin contact between the device and a wearer.

FIG. 5 is an example embodiment of at least a portion of a device of the present disclosure including a mechanism for initiating device start-up and operation when there is adequate skin contact between the device and a wearer.

### DEFINITIONS

Sweat sensor data means all of the information collected by sweat sensing device sensor(s) and communicated via the device to a user or a data aggregation location.

Correlated aggregated data means sweat sensor data that has been collected in a data aggregation location and correlated with outside information such as time, ambient temperature, weather, location, user profile, other sweat sensor data, other wearables data, or any other relevant data.

Chronological assurance means using a sweat sensor device to measure a sweat analyte so that the measurement reflects the analyte's concentration in a fresh sweat sample as it emerges from skin.

By contrast, a sweat analyte measurement lacking chronological assurance may reflect the analyte's concentration in a sweat sample consisting of fresh sweat mixed with older sweat.

Sweat generation rate means the sweat volume per unit time that is produced by sweat glands under or in proximity to a sweat sensor device.

Sweat flow rate means the volume of sweat per unit time flowing across a sweat sensor.

Sensor lifespan means the number of useful readings that a sweat sensor can accomplish for a particular application or the amount of time a sweat sensor can operate on skin for a particular application.

Limited use sensor means a sensor capable of relatively few useful sweat readings such that the sensor must be used only when needed to accomplish a particular sweat sensing device application. For example, a sensor capable of only one, or only a small number of useful sweat readings.

Optimal sensor performance means a set of parameters denoting the best operation of a sweat sensor for a particular application. These include, for example, accuracy, consistency, sensitivity longevity, specificity, selectivity, molar limit of detection, and repeatability.

Minimum sensor performance means a set of parameters denoting the lowest acceptable baseline operation of a sweat sensor for a particular application.

Adequate skin contact means the degree of contact, as measured by an impedance-based skin contact sensor, between a sweat sensor device and a wearer's skin that allows minimum sensor performance.

Adequate skin proximity means the distance, as measured by a capacitive skin contact sensor, between a sweat sensor device and a wearer's skin that allows minimum sensor performance.

Optimal skin contact or proximity means the distance or contact, as appropriate, between a sweat sensor device and a wearer's skin that allows optimal sensor performance.

Power management means the ability to allocate device power in order to: (1) enable a particular device application by managing overall power consumption; or (2) enable device operation by managing real-time power requirements.

### DETAILED DESCRIPTION OF THE INVENTION

The detailed description of the present disclosure will be primarily be, but not entirely be, limited to subcomponents, subsystems, and sub methods of wearable sensing devices, including devices dedicated to sweat sensing. Therefore, although not described in detail here, other essential features which are readily interpreted from or incorporated along with the disclosed invention shall be included as part of the disclosed invention. The specification for the disclosed invention provides specific examples to portray inventive steps, but which will not necessarily cover all possible embodiments commonly known to those skilled in the art. For example, the disclosed invention will not necessarily include all obvious features needed for operation, examples being a battery or power source which is required to power electronics, or for example, an wax paper backing that is removed prior to applying an adhesive patch, or for example, a particular antenna design that allows wireless communication with a particular external computing and information display device. Several specific, but non-limiting, examples can be provided as follows. The invention includes reference to the article in press for publication in the journal IEEE Transactions on Biomedical Engineering, titled “Adhesive RFID Sensor Patch for Monitoring of Sweat Electrolytes”, PCT/US2013/035092, PCT/US14/061083, and PCT/US14/061098, all of which are included herein by reference in their entirety. The disclosed invention applies to any type of sweat sensor device that measures sweat, sweat generation rate, sweat chronological assurance, its solutes, or solutes that transfer into sweat from skin. The present disclosure applies to sweat sensing devices which can take various forms, including patches, bands, straps, portions of clothing, wearables, or any mechanism suitable to affordably, conveniently, effectively, intelligently, or reliably bring sweat stimulating, sweat collecting, and/or sweat sensing technology into intimate proximity with sweat as it is generated. In some embodiments disclosed herein the device will require adhesives to the skin, but devices could also be held by other mechanisms that hold the device secure against the skin such as strap or embedding in a helmet or other headgear. The disclosed invention may benefit from chemicals, materials, sensors, electronics, microfluidics, algorithms, computing, software, systems, and other features or designs, as commonly known to those skilled in the art of electronics, biosensors, patches, diagnostics, clinical tools, wearable sensors, computing, and product design. The disclosed invention applies to any type of device that measures sweat or sweat generation rate, its solutes, solutes that transfer into sweat from skin, a property of or things on the surface of skin, or measures properties or things beneath the skin. The disclosed invention includes all direct or indirect mechanisms of sweat stimulation, including but not limited to sweat stimulation by heat, pressure, electricity, iontophoresis or diffusion of chemical sweat stimulants, orally or injected drugs that stimulate sweat, stimuli external to the body, cognitive activity, or physical activity, or other sweat responses to external stimuli. The disclosed invention includes all mechanisms for determining the device's contact with or proximity to skin, such as impedance electrodes, or capacitive sensors. Any suitable technique for measuring sweat rate should be included in the disclosed invention where measurement of sweat rate is mentioned for an embodiment of the disclosed invention. The disclosed invention may include all known variations of biosensors, and the description herein shows sensors as simple individual elements. It is understood that many sensors require two or more electrodes, reference electrodes, or additional supporting technology or features which are not captured in the description herein. Sensors are preferably electrical in nature, but may also include optical, chemical, mechanical, or other known biosensing mechanisms. Sensors can be in duplicate, triplicate, or more, to provide improved data and readings. Many of these auxiliary features of the device may, or may not, also require aspects of the disclosed invention.

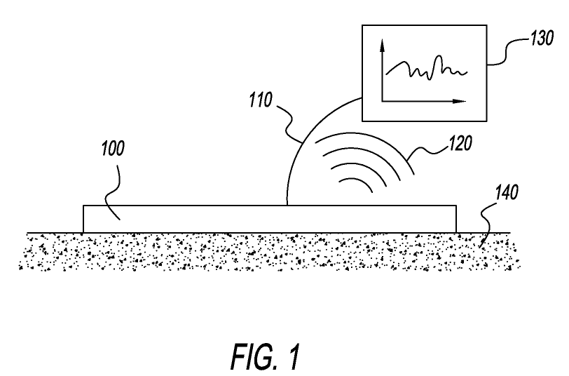

With reference to FIG. 1, a sweat sensing device 100 is placed on or near skin 140, or in an alternate embodiment is simply fluidically connected to skin or regions near skin through microfluidics or other suitable techniques (not shown). A complete enablement of such a device is described by Rose and Heikenfeld in the article in press for publication in the journal IEEE Transactions on Biomedical Engineering, titled “Adhesive RFID Sensor Patch for Monitoring of Sweat Electrolytes”. The disclosed invention applies at least to any type of sweat sensing device that stimulates and/or measures sweat, its solutes, solutes that transfer into sweat from skin, a property of or things on the surface of skin, or properties or things beneath the skin, or measures something about the surrounding environment including humidity, temperature, motion, or other external factors to be measured. Certain embodiments of the present disclosure show sensors as simple individual elements. Certain embodiments of the present disclosure show sub-components of sweat sensing devices that would require additional obvious sub-components for various applications (such as a battery, or a counter electrode for iontophoresis). These additional sub-components are not critical to the inventive step of the present disclosure, and for purpose of brevity and focus on inventive aspects, are not explicitly shown in the diagrams or described in the embodiments of the present disclosure.

With further reference to FIG. 1, the arrangement and description of the device is an example embodiment only, and other obvious configurations and applications are included within spirit of this disclosure. The device 100 is in wired communication 110 or wireless communication 120 with an AC or battery-powered reader device 130, and placed on skin 140. In one embodiment of the present disclosure, the reader device 130 would be a smart phone, or other portable electronic device. In another embodiment, the reader device is a companion transceiver placed at bedside, mounted in a commercial or military vehicle, or widely distributed in locations that are supplied with electrical power. In another embodiment, the reader device is a portable electronic device or companion transceiver capable of secure two-way communication with the sensor and secure two-way communication with a computer network, such as a local area network or the Internet via a wireless router and/or a cellular data network. In alternate embodiments the device 100 and device 130 can be combined (not shown).

The device may include RFID, or may include wireless protocol such as BluetoothTM, or the device may use alternate communication or power strategies to communicate with a reader device in proximity to the device. The sensor can include a thin layer battery and provide its own power source, and thus not rely on RF1D. Both RFID and Bluetooth can be used in conjunction, where RFID can charge the battery when provided the proper near field communications. The device may also include means of signal amplification to improve signal quality communicated to the reader device, and to improve transmission distance to the reader device. Other biomarker sensing methods and sweat transport methods may be included, so long as they provide the same capability of continuous or semi-continuous monitoring of sweat biomarkers.

The sweat sensing device disclosed herein also includes computing and data storage capability sufficient to operate the device, which comprises the ability to conduct communication among components, to perform data aggregation and sensor calibration, to transform raw data into physiologically meaningful information, and to control the sweat sensors and sweat stimulation means, such as iontophoresis electrodes, in real time, or near real time. The device may also employ the capability to monitor and adjust sensor performance in terms of accuracy, sensitivity, consistency, or other relevant factors such expected or known percentage error. Tracking or reporting actual or expected sensor performance degradation may be included as well. The device may also include the ability to monitor power consumption by the sweat sensor device as a whole, or by individual sensors, groups of sensors, communication means, and other individual components. The device may also monitor power available to the device, and compare the power available to power consumption rates to determine an estimated operational duration for the particular application. The disclosed invention may also monitor real-time and anticipated operational power needs, which the device could use to allocate power resources in order to achieve desired performance. This computing capability may be fully or partially located on the sweat sensing patch, on the reader device, or on a connected computer network, including cloud computing.

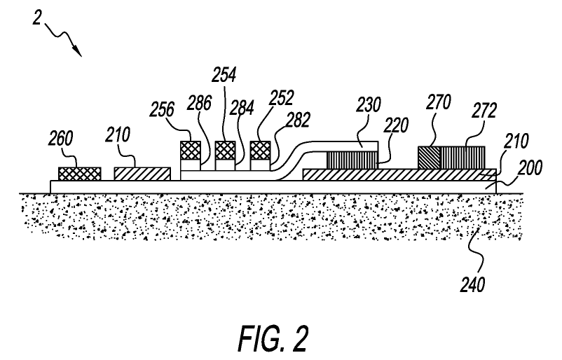

FIG. 2 is an example embodiment of at least a portion of a disclosed device capable of dynamic sensor management. As shown in FIG. 2, a sweat sensing device 2 positioned on skin 240 by an adhesive layer 200 bonded to fluid impermeable substrate 210. Substrate 210 holds electronics 270 (272), one or more sensors 220 (one shown), a microfluidic component 230, coupled to one or more pads 282, 284, 286. Each pad has a source of chemical sweat stimulant, such as pilocarpine, and independently controlled iontophoresis electrode(s) 252, 254, 256. There is also one or more counter electrode(s) 260. The sweat sensor 220 can be a gate-exposed SiCMOS chip having three or more identical chem-FETs per biomarker. Sub-micron SiCMOS allow for MHz impedance spectroscopy. Sensors may be separated spatially into subgroups of identical sensors, or large sensor arrays can be formed using techniques such as photo-initiated chemical patterning. Arrays of such biomarker-specific sensors may allow continuous monitoring of multiple physiological conditions (not shown). Thus, in operation, the electronics 270 (272) would activate one or more iontophoresis electrodes 252, 254, 256. This will cause the skin to generate sweat, which will be transferred through the microfluidic structure 230, and directed to the sensors 220. The sweat sensors 220, in conjunction with sweat stimulation electrodes 252, 254, 256, allow for one-time, intermittent, or continuous monitoring of multiple sweat analytes. In other embodiments, sweat stimulation may be accomplished by means other than iontophoresis, for example, by diffusion of sweat stimulating chemicals into the skin, by increased mental or physical activity by the device wearer, use of exothermic chemical reactions, or a light-based heat source, such as an LED (not shown).

The individual sensors 220 or arrays of sensors may be selectively activated and controlled via a power controller 270 configured to manipulate activation power to the sensors. Such control would allow sensors to be preserved for a single or limited number of uses. For example, the power controller 270 would not send activating power to a limited use sensor, until, for example, a certain amount of time had passed since device activation, after a set time after the occurrence of an event, such as the wearer awakening from a night's sleep, or the detection of a sweat analyte that indicated the need to use the limited use sensor. The power controller may also be used to vary sampling rate. For example, if chronological assurance measurements indicated that sweat generation rate had slowed, and sweat measurements needed to be taken less frequently to ensure measurement of fresh sweat, the power controller 270 could adjust activation timing based on the sweat refresh time calculated from the new sweat generation rate.

Similarly, the power controller may adjust activating power to sensors to provide optimal or minimal sensor performance for the given application, or the specific sensing conditions. As an illustrative example, certain types of sensors 220, such as aptamer-based sensors, are sensitive to the waveform of the driving power. Therefore, adjustments to period, frequency and or amplitude of the driving power can change the performance (i.e. sensitivity, selectivity, limit of detection, gain, accuracy, consistency, and other measures of performance) of those sensors. For instance, if an aptamer sensor used a redox couple, or other pH-sensitive detection technique (i.e., the sensor's peak voltage response changes as pH changes), then the device could employ an ionophore sensor to continuously measure pH in the sweat sample. Then as pH changes, the power controller could correspondingly adjust the peak voltage used for measuring the sensor's detection current or impedance. As another example, a sweat sensor device configured with aptamer sensors to measure cortisol, such as those described in U.S. Pat. No. 7,803,542, may adjust activation power to the sensors based on their sensitivity ranges. Assume a sweat sensing device is configured with two cortisol aptamer sensors. A first sensor may have a sensitivity range of 1 nM to 100 nM, while the second sensor may have a sensitivity range of 100 nM to 10 μM. In order to operate the sensors, the power controller would provide different power profiles to each sensor. The waveform adjustments for aptamer sensors may also account for differences in sweat pH, sweat salinity, sweat generation rate, and sensor temperature, all of which influence performance. The power controller may also account for other factors affecting performance, such as degree of skin contact, sensor degradation, or even the performance characteristics of a particular sensor. In this way, the sensor's performance can be optimized based on sensing conditions.

Further, because such aptamer sensors are likely to require substantial power consumption for operation, the power controller may also activate only relevant sensors, or adjust sampling times to conserve device power. For example, if a sweat sensing device is configured with 3 sets of 5 aptamer sensors, and each set is configured to detect cortisol at distinct, nonoverlapping concentration ranges, the power controller could conserve device power by powering only the set of sensors that corresponds to the detected concentration range, and could reduce sampling frequency from once every 5 minutes to once every 20 minutes.

As with the device's sensors, the power controller may also selectively activate and control iontophoresis electrodes 252, 254, 256 by manipulating activation power. For example, the power controller may activate electrodes 252, 254, 256 to stimulate sweat to an individual sensor or array of sensors at a desired time. To illustrate, a single-use immune-assay sensor for luteinizing hormone (LH) may remain isolated from sweat during device operation until detected estradiol levels indicate an LH would inform a device user whether ovulation was in progress. The power controller would activate an iontophoresis electrode near the LH sensor, stimulating sweat and sending a sweat sample into a microfluidic channel leading across the LH sensor. After a sufficient volume of sweat entered the channel, a barrier dissolves and sweat is able to reach the LH sensor.

Or the power controller may adjust electrode activation power to achieve optimal or minimal sweat rates for a particular sensor or group of sensors. As an example, assume that a sweat sensor capable of detecting cortisol is only able to correlate sweat cortisol to blood concentrations of cortisol at low sweat rates. The sweat sensing device is tasked with measuring cortisol for cortisol awakening response, which occurs roughly 30 minutes after a person awakes from a night's sleep. Prior to the time window for measuring cortisol, the device measures sweat generation rate near the cortisol sensor, and if the sweat rate is insufficient to achieve a meaningful measurement, the power controller could activate an iontophoresis electrode. The activation power timing and voltage would be calculated to provide the needed sweat rate to the cortisol sensor, at the needed time, so that the sweat cortisol measurement can be correlated with blood cortisol concentrations during the window for capturing the cortisol awakening response.

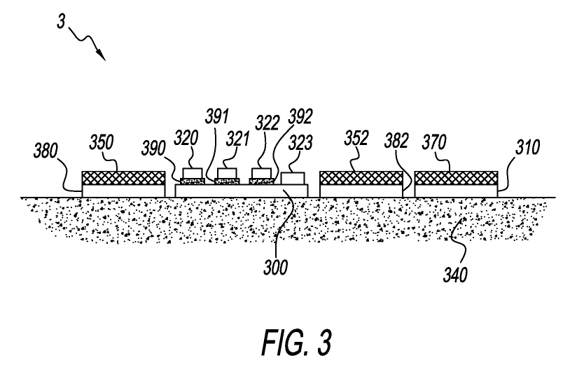

FIG. 3 is applicable to any of the devices of FIGS. 1-2. A particular sensor may need to be isolated from sweat until its use is required. For example, if the sensor is a one-use or other limited use sensor, or if readings from a particular sensor or group of sensors are not needed at device application, or if the use of a sensor is resource intensive in any way, such as in the measure of electrical power or chemicals consumed, then the sensor can remain isolated from sweat until needed. The isolation can be accomplished via selectively porous membrane, gated microfluidic channels, or other suitable means. To cite a previous example, a single-use immune-assay sensor for luteinizing hormone (LH) may be one such sensor that is reserved for one-time or limited use. A sweat sensor device 3 positioned on skin 340 by an adhesive layer 300 carrying three gate components 390, 391, 392 each with at least one sensor 320, 321, 322, and a sensor 323 with no gate component. Electrode 350 is utilized to iontophoretically drive into skin 340 a chemical sweat stimulant suspended in gel 380, with the counter electrode 352 having a gel 382 with no sweat stimulant chemical. Sweat is indirectly induced under the sensors 320, 321, 322, by sudomotor axon reflex sweating as disclosed in U.S. Provisional 62/115,851. Gate components 390, 391, 392 can be any of the numerous gating components known by those skilled in the art of microfluidics, including, for example, pressure actuated gates, electro-wetting, gates created by melting of a polymer or wax, and other suitable techniques. The power controller 370 is positioned on fluid impermeable substrate 310.

Gate components such as 390 could also be a selectively porous membrane material, which could be a material that would not be soluble by sweat, or permeable to solutes in sweat, unless activated by current, voltage, pressure or other stimulus. For example, the selectively porous membrane could be hydrophobic and exhibit the well-known effect of bubble point pressure, which requires a pressure to overcome an initial Laplace pressure as sweat attempts to move through pores in the membrane. Therefore, a sensor such as sensor 320 might not receive sweat unless sweat rate is high enough to enable pressure to permeate gate component 390. Membranes can also be electrically actuated. For example, a membrane material can be configured with nanopores and connected to electrodes that provide the membrane with a surface charge. The membrane then uses Debye electrostatic screening to increase or reduce the permeability of the nanopore in response to a particular charge polarity and/or magnitude. As another example, a nano-porous membrane, such as a track-etch membrane, could exhibit a surface charge in solution that would electrically screen (deplete) charges of the same polarity. This screening would keep some types of charged ions, molecules, proteins, or other charged structures from passing through the membrane. Upon applying voltage across the membrane, the barrier to transport of charged structures through the membrane could be overcome. Such membranes could alternately be constructed from or contain electrodes themselves, and electrically modulate the depth of the charge screening layer inside the pores by depletion or accumulation of charges at the surfaces of the pores. Gate components such as 390 could be gated microfluidic channels, or other suitable means such as electro-wetting gated channels. Any suitable gating mechanism may be used in the disclosed invention with similar effect or cause as described for embodiments of the present disclosure.

With further reference to FIG. 3, sensor 323 could measure sweat rate by impedance or by sodium concentration, for example, in order to determine when sweat rate is at a target level that allows a sweat sensor to take an accurate analyte measurement (e.g., ensuring a sufficiently high sweat rate to counter skin contamination or solute back diffusion, if such issues are of concern; or ensuring sufficiently low sweat rate, if measured analytes are solutes that partition into sweat very slowly, such as proteins). When sweat rate reaches its desired target or target range, a gate component such as 390 could activate, be opened, or otherwise allow sweat transport to a sensor 320.

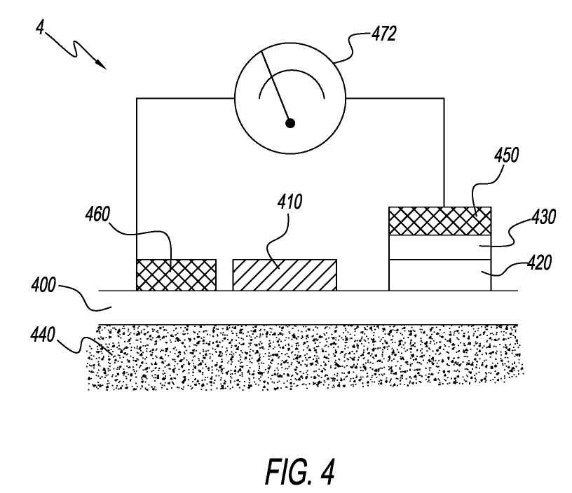

FIG. 4 is applicable to any of the devices of FIGS. 1-3. If electrode/pad contact to the skin is or becomes inadequate, this can be detected as an increase in impedance and the device can adjust power supply to the device or device component, and or alert the user. The sweat sensing device 4 affixed to skin 440 by an adhesive layer 400 bonded to fluid impermeable substrate 410, senses impedance of the contact of the electrode 450 (with chemical stimulant source 430 and microfluidic component 420) with the skin 440 or the contact of counter electrode 460 with the skin 440 where “contact” refers to direct contact, or close proximity or indirect contact that maintains adequate and/or uniform electrical conduction with the skin. Inadequate contact can indicate that the patch become partially or completely detached from the skin. Measurement of electrical impedance includes obvious related measures such as voltage or current, which also give a measure of impedance. If the impedance exceeds a preset limit as measured by circuit 472, the device determines that it is no longer in adequate contact with skin. This preset limit may be correlated with a minimum sensor operation metric to provide an adequate skin contact measurement, or an optimal sensor operation metric to provide an optimal skin contact measurement. The device may include the capability to record and track the time(s) at which a sweat sensor is in contact with the skin, as well as the time(s) at which the sweat sensor is no longer in contact with the skin. The sweat sensing device can be programmed to sense skin contact impedance continuously, or periodically, or upon the occurrence of certain relevant events, such as an increase in natural sweat rate signaling increased physical activity.

In other embodiments, the device 4 may be configured with two or more skin facing electrodes dedicated to determining skin and/or body impedance (not shown), as are known to those skilled in the art of electrophysiology. Similarly, in other embodiments, at least one capacitive sensor electrode (not shown), also as known in the art of electrophysiology, may be placed on selected locations on the skin-facing side of the device, and would convey information about the distance between the capacitive sensor and the skin. The skin proximity measurement produced by the electrodes could be an adequate skin proximity metric correlated with a minimum sensor performance, or an optimal skin proximity metric correlated with an optimal sensor performance. The skin proximity readings generated by the capacitive sensor(s) would therefore indicate whether the device is in optimal, adequate or inadequate proximity with a wearer's skin.

The device may use such skin contact readings for a number of purposes. For instance, the device may be configured to execute a start-up sequence whereby prior to application of the patch to a wearer, the device periodically checks for skin contact until the patch is applied to skin and skin contact is detected, or the device may initiate a start up sequence upon the removal of a protective film, or other such suitable means. Once in good contact with skin, the device would then perform certain initialization functions, such as establishing communication between components, initiating safety or compliance checks, assessing device operation, performing sensor calibration, configuring the device for operation, stimulating sweat to wet sensors prior to use, or other functions. During operation, skin contact measurements may be used to adjust power allocation to sensors and iontophoresis electrodes to manage power consumption and device performance. For example, a sweat sensor device configured with capacitive sensors may activate such sensors when a protective backing is removed from the skin-facing adhesive. When the device is applied to skin and the capacitive sensors detect proximity (e.g., within 100 μm) to a wearer's skin, the device conducts an initialization protocol to prepare the device for use. The power controller activates the capacitive sensors periodically, e.g. every 5 minutes, and two hours into device operation, the capacitive sensors measure device-skin proximity which could be for example ˜1000 μm. At this point, the affected sweat sensor will no longer perform meaningful measurements, i.e., is no longer capable of minimum performance. The power controller may then deactivate the affected sensors and iontophoresis electrodes.

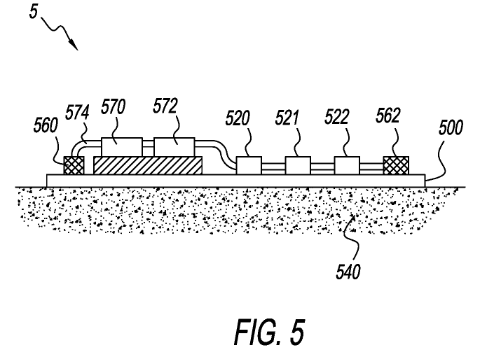

FIG. 5 is applicable to any of the devices of FIGS. 1-4. In another disclosed embodiment, the device power controller 570 may be integrated into a circuit with wires or communication bus 574 that requires skin contact to initiate or maintain operation. The sweat sensing device 5 affixed to skin 540 by adhesive 500, as shown in FIG. 5, is powered by a power source such as a battery (not shown) connected to the power controller 570. The power controller 570 is in a circuit through wires 574 with two electrodes 560, 562. The power controller 570 has little or no current flow between electrodes 560 and 562 until electrodes 560 and 562 are placed in adequate contact with skin via adhesive 500. Once the electrodes 560 and 562 are in contact with skin, the power controller 570 energizes the other device components, including powering of additional controllers or electronics 572 and one or more sensors 520, 521, 522 to initiate device power-up and enable the system to perform initialization and operation. The power controller may be configured to bypass the start-up circuit after initialization to allow operation without having a completed start-up circuit. Alternatively, the power controller may supply power only as long as the start-up circuit remains complete. Startup can be initiated through numerous sensors and means that correspond with application of the sweat sensor device 5 to skin 540, including even removing of sweat sensing device 5 from its packaging (not shown) which is effectively also at or near the time of placement on skin 540.

The sweat sensor data monitored by the user may include real-time analyte concentration, sweat pH, sensor temperature, sweat flow rate, analyte to analyte ratios, analyte concentration or ratio trend data, or may also include aggregated sweat sensor data drawn from a database and correlated to a particular user, a user profile (such as age, gender or fitness level), weather condition, activity, combined analyte profile, or other relevant metric. Such data aggregation may include collecting and incorporating sweat sensor performance data, sweat rate, sensor power consumption, skin contact/proximity, or other relevant information generated by a device. The sweat sensor data may also be correlated with outside information, such as the time, date, weather conditions, activity performed by the individual, the individual's mental and physical performance during the data collection, the proximity to significant health events experienced by the individual, the individual's age or sex, the individual's health history, data from wearable devices or sensors, such as those measuring galvanic skin response, pulse oximetry, heart rate, etc., or other relevant information. Particular to sensor management capabilities, outside information may also include expected time intervals between a physiological event and the indication of that event in sweat, average power requirements for particular types of equipment, average lifespan for particular sensor types, optimal power levels for particular sensors under various conditions, sensor calibration factors (such as performance, remaining sweat stimulant amounts available to iontophoresis electrodes, remaining capacity in waste sweat reservoirs, or participation by the device wearer in activities that tend to dislodge patches from skin contact, among other things).

Correlated aggregated data would allow the user to compare real-time sweat sensor performance or power consumption to external data profiles for the sensor, or corresponding sensors or sensor types. For example, a external data profiles assembled for aptamer cortisol sensors may include profiles for sensor calibration and optimization. For example, every sensor could be encoded with performance and calibration data, so that the device could determine the best waveform and calibration for the sensor. Further, the power controller may compare real-time sweat sensor performance or power consumption to historical performance data for corresponding sensors or sensor types under similar conditions. For example, on a device configured to monitor ovulation via an aptamer-based estradiol sensor, the power controller may anticipate optimal performance power levels, or error-check performance metrics by accounting for performance data on similar estradiol sensors under a similar sweat flow rate, sweat pH, sensor temperature, or other metric. These disclosed uses of aggregated data are for illustration purposes only, and do not limit other potential sources or applications available for such data, which are within the spirit of the present disclosure.

The disclosed invention may be configured to manage the lifespan of the sensors on a sweat sensor device. Using a sweat sensor may tend to degrade its performance for various reasons, including the type of analyte it detects, the method of detection, or contamination from substances in sweat. It therefore may be advantageous to minimize the use of a particular sensor while adequately performing the sensor's desired function. For example, the sweat sensor device may perform periodic sensor quality assessments on a particular sensor. If a sensor indicated it was approaching the end of its usable life, the device could reduce the sampling rate of that sensor to maximize lifespan, or to preserve the sensor for a time when its function would be more critical. To return to the previous cortisol aptamer sensor example, rather than rationing measurements based on historical performance, the power controller may instead determine that sensor output is migrating toward the outer limits of a set acceptable range and reduce or cease its use accordingly in order to cover critical sensing periods, such as during the diurnal cortisol trough window. Similarly, a sensor could be electrically activated or microfluidically connected to sample sweat at a reduced cycle even from the onset of sensing (e.g., to increase its lifespan or reduce data output) and later, if higher resolution (shorter time intervals of measurement) is needed, the sensor would then be utilized more frequently. For the cortisol example, the power controller may be programmed to perform only a minimal number of cortisol readings during the day, while sampling at the maximum chronologically assured rates for the trough and peak windows—even if historical data indicated that such sensors had more available uses, and real-time performance remained optimal.

A plurality of one-time use sensors could be used similarly to a single sensor with a plurality of accurate uses. For example, a device configured to predict ovulation could have 4 LH optical immunoassay sensors, each of which is only capable of one use. The power controller may activate one of the sensors each time one is needed for the particular application, up to a maximum of four uses.

For most applications, the device would likely need to perform at least one measurement that allows the device or user to assess and control how often to employ a one-use or limited-use sensor. This need is particularly important for one-time sensors configured to detect ultra-low concentration biomarkers (nM to pM) using sensing techniques such as chemiluminesence, electrical impedance spectroscopy, antibody, and aptamer-based sensors.

To increase sensor lifespan, or to preserve a limited-use sensor for a needed circumstance, it may also be advantageous to prevent a particular sensor from having contact with sweat via various means. For example, when a sweat sensor device is first applied to a user's skin, a limited-use sensor could be sealed off from sweat via a membrane. The membrane could be electrically activated, for example by applying current, as discussed earlier in this disclosure, to allow sweat to flow to the sensor at the required time. A sensor may also be kept away from sweat via microfluidic manipulation of sampled sweat fluid, for example by using gated microfluidic channels or components.

Another disclosed embodiment could be configured to manage the power consumption of a sweat sensor device. Using a sensor or other component consumes power, which reduces battery life (if a battery is used) and takes operational power resources that otherwise would be available for other functions. Power management, therefore is another reason it may be advantageous to minimize sensor and other component use within performance requirements. A sweat sensor device with chronological assurance capability could determine the maximum meaningful sweat sampling rate, and correspondingly only activate a sensor or group of sensors when a meaningful reading could be taken. For example, if the maximum chronologically assured sweat sampling rate were once every 10 minutes, the device could activate selected sensors at 10-minute or longer intervals to reduce power use and still get meaningful data. Similarly, the sweat sensor device could account for the relative power requirements of a sensor or group of sensors when selecting a sampling interval. For example, if a particular sensor, such as an aptamer-based sensor, required relatively more power to operate, the sweat sensing device could activate the sensor less frequently. Likewise, if the device detected a malfunctioning sensor, or a spent limited-use sensor, the device could stop activation current to that sensor.

In addition to effectively managing overall power consumption, it may also be advantageous to manage real-time power requirements during device operation. At any given moment, a sweat sensing device will have limited power resources to allocate to various functions, which may include, without limitation, sweat sensing, sweat stimulation, and communication to and from the device. The sweat sensing device may therefore account for real-time power requirements to manage the timing or to adjust the activation power applied to sensors, sweat stimulation, or communication. For example, a sweat sensing device may detect elevated levels of K+ indicating muscle damage, and algorithms interpreting the data correspondingly instruct a suite of sensors capable of detecting Rhabdo biomarkers, and their corresponding iontophoresis electrodes, to activate. The increased power needs of the Rhabdo sensors and electrodes could then prompt the device to delay a scheduled data upload until the specialized sensors had conducted their reading, thereby not exceeding the power available for device operation. In another example, aptamer sensors and other sensors employing a driving waveform, require orders of magnitude more power to operate than do potentiometric sensors, such as ISE's. Therefore, the device could activate aptamer sensors less frequently than it activates lower power ISE sensors. In another illustrative example, a sweat sensor device may employ a plurality of sensor groups that perform the same or similar functions. During operation, the sweat sensor device may compare data from the sensor groups. If one of the groups produces divergent data, for example because the group was malfunctioning, or was not exposed to sweat, the power controller could stop activating the divergent group to conserve power.

The sweat sensing device may also be equipped to provide optimal or minimum performance by a sensor or group of sensors. A number of conditions may impact sensor performance, both within and outside the sensing environment, that the power controller may have to address in order to achieve optimal or minimum acceptable sensor performance.

For example, sweat rate affects solute concentration in sweat, causing some analytes, such as Cl— to increase in concentration with increased sweat rate, and causing others, such as proteins, to decrease in concentration with increased sweat rate. Sweat pH has a significant effect on ionophore sensor performance, greatly influencing the binding affinity of such sensors to their target analytes, and thus influencing sensor sensitivity. The temperature of sensors also affects sensor performance, for example, by affecting the thermodynamic equilibrium of the system, as described in the Nernst equation, which can be used to characterize the response slope of an ISE with respect to a change in target ion concentration in sweat. Further, sensors are subject to manufacturing variabilities, which cause them to respond differently than other sensors to similar sensing conditions. The typical size or concentration of a target analyte in sweat may also affect sensor performance. The sweat sensor device may perform differently due to placement on the body of a wearer, or due to the adequacy of skin contact. Further, sweat sensor performance may degrade during operation due to sensor fouling caused by prolonged contact with sweat samples. In addition to algorithmic data correction, some of these performance issues may be managed through power adjustments to the sensors themselves, while other variables may be managed by adjustments to sweat rate.

The different types of sensor, such as ionophore, amperometric, or aptamer sensors, have different ideal and minimal performance environments. For example, the power controller could adjust activation power to an impedance sensor to improve its performance during periods of high sweat rate. The conductivity of the sweat sample typically increases with sweat rate, which would, in turn, decrease the shunt resistance for an impedance-type sensor such as those used in electrical impedance spectroscopy. Therefore, to improve the performance of the impedance sensor, the sampling frequency could be increased so that the sensed impedance signal (such as electrical capacitance) increases relative to the background impedance signal (caused at least in part by shunt resistance). To account for manufacturing variances, for instance, the power controller could adjust a sensor's activating power based on an initial device or sensor calibration. As another example, for amperometric or other sensors that consume the analyte during the measurement process, sweat sampling rate may be correlated to the sweat generation rate. For higher sweat generation rates (>1 nL/min/gland), amperometric sensors should be operated at increased power, or at higher sampling rates, while at lower sweat generation rates (<0.5 nL/min/gland), such sensors should be operated at lower power, or should sample less frequently. This technique will ensure that the analyte concentration measured by the sensor will have a stronger signal than the background noise affecting the sensor. To cite another example, aptamer-based sensors may only be sensitive within a limited range of analyte concentrations. The sweat sensor device may accordingly be configured with a plurality of aptamer sensors that have different sensitivity ranges corresponding to different sweat sample concentrations. The power controller may therefore activate only those aptamer sensors with a sensitivity range corresponding to the sweat sample concentration, thus limiting power consumption, improving sensor lifespan, and improving sensor performance.

In addition to, or instead of, adjusting activation power to device sensors to provide optimal or minimum sensor performance, the power controller may be configured to adjust sweat rate. Stimulating sweat via iontophoresis or electrosmosis may be used to manage the use of a particular sensor or sensor suite by actively influencing the sweat flow rate to that sensor. For example, a sensor operating under certain conditions may require a higher sweat rate for optimal sensitivity or accuracy, and the device could increase sweat stimulation to provide the necessary sweat rate. Similarly, a sensor that is already being supplied with sweat via stimulation may require a lower sweat rate for optimal operation. The power controller could accordingly reduce power to iontophoresis electrodes to cause the sweat rate to decrease. In another example, a specialized, or limited-use sensor may be required to detect a certain analyte. Upon the occurrence of a trigger event indicating that the analyte may be present in sweat, and after a calculated time interval has elapsed, the device could initiate sweat stimulation to induce sweat flow to the specialized sensor at the desired time. As another example, a sensor configured to detect larger molecules in sweat, such as proteins, peptides, or hormones, may require a low sweat rate to ensure sweat concentrations of these analytes correlate with blood concentrations (such molecules diffuse slowly from blood into sweat, and therefore tend to drop in sweat concentration as sweat rate increases, becoming decoupled from blood concentrations). The power controller may accordingly reduce sweat stimulation power in the proximity of the specialized sensor to ensure sweat and blood concentrations remain correlated.

By adjusting activation power and or sweat rate, the sweat sensor device may also clean, de-foul, or otherwise regenerate sensors to improve performance. For example, if an ionophore sensor's performance became degraded during operation due to ions adhering to the sensor, the power controller could drive cyclic voltammetry modulated current into the sensor to drive off the adhering ions. Once cleaned, the power controller would resume supplying normal operating power to the sensor. Another method available to de-foul ionophore sensors is through local changes to sweat sample pH. For instance, pH may be altered by activating an iontophoresis electrode upstream of a particular sweat sensor. By driving ions off the electrode and into the sweat sample, the H+ concentration level in the sweat sample can be altered, which in turn causes ions adhering to the downstream sensor to return to solution. For example, if an AgCl ionophore sensor configured to detect sweat Cl— became fouled with adhering Cl— ions, the power controller could send activating current into an iontophoresis electrode upstream of the sensor. The current would cause H+ to detach from the electrode and enter the sweat sample, thereby lowering pH. As the sweat sample continues past the AgCl sensor, it pulls Cl— ions off the sensor and into the sample to bind with the H+ ions.

Biorecognition sensors may also be cleaned by exposing them to sweat samples generated at high sweat generation rates. Because of relatively slow partition into sweat, larger molecules like proteins, peptides and nucleases become diluted in sweat at higher sweat generation rates. These types of analytes are detected by using an immunoassay, an aptamer sensor, electro-impedance spectroscopy, or other biorecognition-based sensor. Reducing the concentration of such analytes in sweat will cause analytes bound to sensor biorecognition elements to disassociate and return to solution. Therefore, if larger analyte sensors become fouled by analytes, exposing those sensors to high sweat rates will tend to wash or refresh the sensors. High sweat generation rates may be developed in proximity to a sensor or group of sensors by disclosed sweat stimulation methods to increase local sweat generation. Once the device washes the sensors, it may then resume sweat measurements, for example after delaying a set time period, or after measured sweat generation rates have returned to pre-stimulation levels.

Another embodiment disclosed herein may be configured to perform dynamic analyte detection. A sweat sensor device may be deployed carrying different types of sensors optimized for detecting different analytes. Due to a number of factors, including sensor lifespan, power consumption needs, data volume control, data security, or the wearer's physical condition, it would be advantageous to be able to activate a specialized sensor or sensor suite only when needed. The need to take measurements with such specialized sensor(s) may be based on the occurrence of a particular event, or the existence of defined conditions. For example, a first sensor might continuously monitor a certain analyte, while the remainder of the device's sensors remain deactivated. If readings by the first sensor indicate a condition is occurring or may occur soon, but alone those readings are insufficient to make a conclusive determination, the device could then activate an additional sensor or sensors to monitor more directly or quantitatively that condition. For example, a device continuously monitors ammonia using a set of long-lifespan sensors. During the monitoring period, ammonia levels reach a threshold indicating that a possible heart attack is in progress. The device could then activate a specialized limited lifespan sensor to detect biomarkers associated with cardiac distress, such as natriuretic peptides, troponin or creatine kinase-MB. In another example, a device might continuously monitor K+ for signs of muscle damage, then if K+ readings reach a threshold value, the device could activate sensors capable of detecting Rhabdo biomarkers to confirm that muscle damage has occurred. Similarly, for a sweat sensor device configured to detect when a wearer is dehydrated, the device may continuously measure sweat generation rate by using a galvanic skin response sensor, or by measuring the ratio of sweat sample concentrations of Cl— to K+. When device measurements indicated a sharp increase in sweat rate, or a sustained elevated sweat rate, the power controller could activate a limited use sensor configured to detect vasopressin. Elevated vasopressin levels coupled with water loss would indicate that the wearer had entered a state of dehydration.

Using correlated aggregated sweat sensor data, the sweat sensing device may calculate a time interval after an event when a desired analyte is expected to appear in eccrine sweat. When the time interval has elapsed and the target analyte is expected to appear, the device could activate specialized sensors. For example, after a sensor detects elevated K+ levels in sweat, there is a measurable delay after the occurrence of the event until Rhabdo biomarkers emerge and become optimally detectible in sweat. The device could analyze correlated and aggregated sweat sensor data to calculate the expected time interval for an individual based on relevant factors such as age, fitness, weight, individual history, or other relevant factors. Based on this calculation, the device could preserve the specialized sensors capable of detecting Rhabdo biomarkers until the calculated interval has elapsed, thus improving the device's ability to make a meaningful reading.

In another example, the sweat sensing device may determine sweat generation rates in proximity to a particular sensor or sensor suite, and only activate those sensors when the sweat generation rate will allow a meaningful reading. Thus, if the sweat generation rate were too high to allow sweat concentrations of a protein to correlate with blood concentration, the sweat sensing device could delay sensor activation until a lower sweat generation rate were achieved.

Finally, a particular sensor or sensor suite may have a function such that the information they would generate would be redundant, or unnecessary for a particular application, or only meaningful given the occurrence of a physiological event. In these cases, the device would not activate such sensors unless or until they were needed. As an example, the sweat sensor could use inputs from an accelerometer to determine when to take sweat measurements. For instance, if a sweat sensor were in use for monitoring an elderly wearer who is prone to falls, the sweat sensor could receive data from an accelerometer that indicates the wearer is ambulating, causing the sweat sensor to activate and take measurements. For athletes, an accelerometer may indicate prolonged physical activity that prompt the sweat sensing device to activate sensors to determine hydration levels. Similarly, a wearer may be monitored for alcohol use with a sweat sensing device that activates ethanol sensors upon input from an accelerometer indicating reduced coordination, or a GPS device indicating vehicle operation.

In another embodiment of the disclosed invention, the sweat sensing device would be configured to manage skin contact issues. If a sensor starts to come loose from the skin, it will have less contact with skin or sweat, and therefore would experience altered operational performance. By sensing electrical impedance between the sensor and the skin (less contact=higher electrical impedance), the amount of skin contact by the sensor could be determined. Alternately, capacitive sensors could be used to provide skin proximity measurements. The device could then accordingly adjust the driving frequency or amplitude, or other waveform features, applied to the sensor in order to enable operation, or improve accuracy/sensitivity given the degree of skin contact. If skin contact were sufficiently degraded to prevent accurate function, the device could deactivate the affected sweat sensors and sweat stimulation electrodes, thus reducing power consumption. The power controller could then shift power to other sensors and electrodes that are operational, or more fully operational.

The following examples are provided to help illustrate the present disclosure, and are not comprehensive or limiting in any manner. These examples serve to illustrate that although the specification herein does not list all possible device features or arrangements or methods for all possible applications, the invention is broad and may incorporate other useful methods or aspects of materials, devices, or systems or other embodiments, which are readily understood and obvious for the broad applications of the present disclosure.

### EXAMPLE 1

A patient is undergoing clinical trials for a new oncology drug. Based on a testing profile developed for the trial, the device has been configured to near-continuously monitor a set of three analytes whose relative concentrations in sweat and concentration trends indicate with reasonable certainty that the patient is taking the drug. The presence of a fourth analyte in sweat would confirm that the patient has taken the drug, however, the specialized sensors necessary to detect the analyte are one-use sensors. The device therefore also includes a limited number of the one-use sensors. Each one-use sensor is isolated from sweat via a selectively permeable membrane. When the multi-use sensors indicate that the drug has been taken, the device waits a calculated interval, and then activates an electrode near an unused one-use sensor, causing the membrane to open and inducing sweat flow to the sensor. The device then activates the one-use sensor, which detects the confirming analyte. Once the reading is recorded, the device stops activation current to the one-use sensor and its iontophoresis electrode.

### EXAMPLE 2

A cyclist is competing in a multi-hour stage of a multi-stage race. Estimated battery life for the sweat sensor device is projected to cover the entire race day. Upon initial application of the sweat sensor device, the device conducts a calibration routine, which determines that the device is in good contact with the skin for proper operation, and calculates optimum and minimum activation currents and voltage for the main type of sensors, which are configured to detect K+. During the race, the device conducts regular power consumption measurements, and determines that power consumption is greater than anticipated and that device battery power is no longer projected to last the entire stage. The device also conducts a chronological assurance reading, which finds that the minimum time between assured sweat readings is 10 minutes. The device accordingly ensures the K+ sampling interval is greater than the 10 minute minimum, stops activation current to a portion of the K+ sensor suite, and, for the remaining K+ sensors, reduces activation current to the minimum operating current and voltage. The device's battery power is now projected to last the entire stage.

### EXAMPLE 3

Continuing the scenario in Example 3, during the bicycle race stage, the device conducts a number of readings, including skin contact readings, to assess why device battery life is shorter than expected. The device discovers that a group of 3 sensors is no longer in adequate contact with skin, and is using extra power. The device accordingly stops activation current to, and, if applicable, iontophoresis activation current corresponding to, the loose sensors. Later during the stage, the device detects elevated K+ levels, and overriding power conservation measures, temporarily increases activation current for the operational K+ sensors to optimum levels, and stimulates sweat for a confirmatory reading. Using correlated aggregated sweat sensor data, the device confirms that K+ levels have exceeded a threshold for the wearer indicating muscle damage. The device also uses correlated aggregated sweat sensor data to calculate when Rhabdo biomarkers are expected to appear in Eccrine sweat for this wearer, under current conditions. After the calculated interval has elapsed, the device activates a group of one-use sensors configured to detect Rhabdo biomarkers. The device exposes the isolated Rhabdo sensors to sweat, and takes a reading confirming muscle damage. After completing the reading, the device reassesses battery life, and then reconfigures the device to conserve power.

### EXAMPLE 4

A child with Type I diabetes is prescribed to wear a sweat sensing device at night. The sweat sensing system consists of a kit containing a number of devices configured for monitoring conditions of hypoglycemia via the amounts and ratios of glucose and at least one other relevant analyte, such as cortisol, detected in sweat. The kit also contains a bedside transceiver, which is in wireless communication with the child's parents' smartphones via the Internet. At bedtime, a device is placed on the child's skin. Upon application, the device performs a start-up sequence, initial calibration, and establishes communication with the bedside transceiver, which sends a status message that the system is fully operational to the parents' smartphones. After taking an initial hypoglycemia reading and finding it normal, the device establishes an initial testing interval of 15 minutes. Three hours later, the device conducts a routine hypoglycemia reading, which indicates a downward trend for glucose and an upward trend for cortisol that exceeds a preset threshold. The system also registers a slight increase in sweat rate. The system enters a first-stage escalation in which the sweat sampling rate is increased to determine if a hypoglycemic state is imminent. After an additional 10 minutes of increased-rate sampling, the system determines that the child is entering a hypoglycemic state, and generates an alert message to the parents' smartphones. The parents are awakened and administer oral glucose tablets to restore the child's blood glucose levels.

This has been a description of the disclosed invention along with a preferred method of practicing the invention, however the invention itself should only be defined by the appended claims.

1. A sweat sensing device configured to be worn on an individual's skin and to perform dynamic sensor management, comprising:
at least one sweat sensor to provide one or more measurements of sweat;
at least one skin proximity sensor;
a communication means; and
a power controller, where the power controller permits a power source to supply electrical power to at least one device component based on at least one measurement by the proximity sensor that indicates adequate proximity between the device and skin.

2. The device of claim 1 further including at least one sweat stimulation pad.

3. The device of claim 1 where at least one sensor measures sweat generation rate.

4. (canceled)

5. The device of claim 1 in which an incomplete start-up circuit connects a power source to the device and the power controller permits the power source to supply electrical power to the device when the start-up circuit is completed by skin contact.

6. (canceled)

7. The device of claim where the power controller is capable of controlling activation power to the at least one sweat sensor to adjust sweat sampling rate.

8. The device of claim 3 where the power controller is capable of controlling activation power to the at least one sweat stimulation pad to adjust sweat generation rate.

9. The device of claim 1 where the power controller is capable of controlling activation power to the at least one sweat sensor based on the useful lifespan of the sweat sensor.

10. The device of claim 9 where the power controller performs periodic assessments to determine the remaining useful lifespan of the at least one sweat sensor.

11. The device of claim 9 where the power controller is capable of controlling activation power to a plurality of sweat sensors to manage remaining sweat sensor lifespan.

12. The device of claim 1 where the power controller performs periodic assessments to determine the at least one sweat sensor's operational functionality.

13. The device of claim 1 where the power controller determines an optimal activation rate and a minimum activation rate for the at least one sweat sensor.

14. The device of claim 3 where the power controller determines an optimal sweat generation rate and a minimum sweat generation rate for the at least one sweat sensor.

15. The device of claim 1 where the power controller is capable of controlling activation power to the at least one sweat sensor to adjust sweat sensor data generation.

16. The device of claim 2 where the power controller is capable of controlling activation power to the at least one sweat stimulation pad to increase a sweat generation rate in proximity to a plurality of sweat sensors.

17. The device of claim 2 where the power controller is capable of controlling activation power to at least one of the following device components in order to manage device power consumption: a sweat sensor, a sweat stimulation pad, and a communication means.

18. The device of claim 2 where the power controller is capable of controlling activation power to at least one of the following device components in order to manage operational power use: a sweat sensor, a sweat stimulation pad, and a communication means.

19. The device of claim 1 where the power controller is capable of controlling activation power to at least one device component to manage at least one of the following: device power consumption, operational power use, device operational duration, and quantity of data output.

20. The device of claim 1 where the power controller activates at least one limited use sweat sensor only when data from the sensor is needed by a device user.

21. The device of claim 20 where the limited use sweat sensor is isolated from sweat by one of the following: a selectively operable gate and a selectively operable membrane.

22. The device of claim 20 where the power controller determines that data from the limited use sensor is needed by the user based on a least one measurement from the at least one sweat sensor.

23. The device of claim 20 where the power controller activates the limited use sensor after the occurrence of an event and after the sensor's target analyte will be detectible in sweat.

24. The device of claim 1 where the at least one sweat measurement is aggregated with other sweat sensor data and correlated with relevant data external to the sweat sensing device, and used to enhance the power controller's management of device operation.

25. (canceled)

26. A method of controlling power to at least one first sweat sensor sensing device component based on the device's proximity to a wearer's skin, comprising:
taking at least one measurement with a skin proximity sensor;
comparing the measurement to a threshold value indicating an adequate proximity to skin;
providing power to the first device component if the measurement indicates adequate proximity to skin; and
removing power to the device component if the measurement indicates an inadequate proximity to skin.

27. (canceled)

28. The method of claim 26 where the method further includes comparing the measurement to a threshold value indicating a sub-optimal proximity to skin; and
adjusting power to the first device component if the measurement indicates sub-optimal proximity to skin.

29. The method of claim 28 where the method includes adjusting power to a second device component if the second device component is in adequate proximity to skin.

30. (canceled)

31. A method of power consumption management for a sweat sensing device, comprising:
determining a remaining operation time required for a sensing device to perform a device user's purpose;
determining a total electrical power requirement of a plurality of device components needed to perform the purpose;
determining an available total power requirement for the sensing device;
comparing the components' power requirement to the available power; and
adjusting power provided to the components to allow operation for the remaining required operation time.

32. The method of claim 31 where a sweat sampling rate for at least one sweat sensor is adjusted.

33. The method of claim 31 where at least one component's power requirement is determined using aggregated sweat sensor data correlated with relevant data external to the sweat sensing device.

34. A method of optimizing performance of a sweat sensor, comprising:
assessing the sensor's performance using a plurality of metrics including accuracy, sensitivity and consistency;
adjusting the power provided to the sweat sensor to adjust sweat sampling rate; and
adjusting sweat generation rate in proximity to the sweat sensor to allow optimal sensor performance.

35. The method of claim 34 where the sensor's performance is determined using aggregated sweat sensor data correlated with relevant data external to the sweat sensing device.

36. A method of dynamic analyte detection by a sweat sensing device, comprising:
using at least one sweat sensor to take at least one measurement of a first analyte in sweat; and
using the at least one measurement to determine that a device user's application requires the measurement of at least one second analyte; and
activating at least one limited use sensor to detect the second analyte.

37. The method of claim 36 where the device delays activation of the limited use sensor until after the second analyte is likely to appear in a sweat sample.

38. The method of claim 36 where the device isolates the limited use sensor from sweat until the limited use sensor is activated.

39. The method of claim 36 where the device controls a sweat flow rate to the limited use sensor by controlling sweat generation rate in proximity to the limited use sensor.

40. The method of claim 36 where the device uses aggregated sweat sensor data correlated with relevant data external to the sweat sensing device to perform dynamic analyte detection.

41. The method of claim 26, further comprising:
performing a plurality of sweat sensing device initialization functions, where the initialization functions include at least one of the following: establishing communication between a first device component and a second device component; performing at least one check to determine wearer compliance; assessing the operational quality of at least one device component; calibrating at least one sweat sensor; and stimulating sweat production to cause a sweat sample to wet a sweat sensor prior to the sweat sensor's use.

42. (canceled)
